(19) United States
(12) Patent Application Publication
(10) Pub. No.: US 2024/0075109 A1
(43) Pub. Date: Mar. 7, 2024

(54) TELOMERASE REVERSE TRANSCRIPTASE-BASED THERAPIES

(71) Applicants: Fundación del Sector Público Estatal Centro Nacional de Investigaciones Oncológicas Carlos III, Madrid (ES); Universitat Autónoma de Barcelona, Bellaterra (ES)

(72) Inventors: Maria Bobadilla, Rosenau (FR); Ivan Formentini, Hovås (SE); Maria Antonia Blasco Marhuenda, Madrid (ES); Christian Baer, Hannover (DE); Fatima Bosch Tubert, Ballaterra (ES)

(21) Appl. No.: 18/463,155

(22) Filed: Sep. 7, 2023

Related U.S. Application Data
(63) Continuation of application No. 17/189,368, filed on Mar. 2, 2021.
Application No. 17/189,368 is a division of application No. 15/502,522, filed on Feb. 8, 2017, now Pat. No. 11,529,396.

(30) Foreign Application Priority Data
Aug. 8, 2014 (EP) 14382312.8

(51) Int. Cl. A61K 38/45; A01K 67/027; A61K 48/00; C12N 9/12

(52) U.S. Cl. CPC A61K 38/45; A01K 67/0271; A01K 67/0276; A61K 48/005; A61K 48/0058; A61K 48/0066; C12N 9/1276; C12Y 207/07049; A01K 2207/12; A01K 2217/075; A01K 2217/206; A01K 2227/105; A01K 2267/03; A01K 2267/0381; C12N 2750/14145

(57) ABSTRACT

The invention provides compositions and methods useful for the treatment and prevention of conditions associated with short telomere length.

### REFERENCE TO AN ELECTRONIC SEQUENCE LISTING

The contents of the electronic sequence listing (370106_401C2_SeqListing.xml; Size: 46 kilobytes; and Date of Creation: Sep. 6, 2023) is herein incorporated by reference in its entirety.

### FIELD OF INVENTION

This invention falls within the field of molecular biology, biotechnology and medicine. More particularly, it relates to compositions and methods useful for the treatment of conditions associated with short telomere length. More particularly, it relates to compositions and methods useful for the treatment of conditions associated with aplastic anemia.

### BACKGROUND OF THE INVENTION

Telomeres are specialized structures at the ends of chromosomes, which have a role in protecting the chromosome ends from DNA repair and degrading activities (Blackburn, 2001. Cell 106, 661-673; de Lange, 2005. Genes Dev. 19, 2100-2110). Mammalian telomeres consist of TTAGGG repeats bound by a multi-protein complex known as shelterin (de Lange, 2005. Genes Dev. 19, 2100-2110). A minimum length of TTAGGG repeats and the integrity of the shelterin complex are necessary for telomere protection (Blackburn, 2001. Cell 106, 661-673; de Lange, 2005. Genes Dev. 19, 2100-2110). Telomerase is a cellular reverse transcriptase (TERT, telomerase reverse transcriptase; also known as TP2; TRT; EST2; TCS1; hEST2) capable of compensating telomere attrition through de novo addition of TTAGGG repeats onto the chromosome ends by using an associated R A component as template (Terc, telomerase RNA component) (Greider and Blackburn, 1985. Cell 43, 405-413). Telomerase is expressed in most adult stem cell compartments, however, this is not sufficient to maintain telomere length as evidenced by the fact that telomere shortening occurs with age in most human and mouse tissues (Harley et al., 1990. Nature 345, 458-460; Blasco, 2007. Nat Chem Biol. 3, 640-649; Flores et al, 2008. Genes and Dev 22, 654-667).

Mice carrying homozygous deletion for the TERC gene (the telomerase RNA component) lack any detectable telomerase activity and showed progressive telomere shortening from one generation to the other at a rate comparable to the rate reported in human cells (Blasco et al., 1997). Severe phenotypes typical of late generation TERC−/− mice (e.g. bone marrow aplasia and signs of premature aging) could be rescued by re-introducing a copy of the TERC gene (Samper et al., 2001). Multiple tissue degeneration arising in later generations in a conditional mouse model defective for TERT (the catalytic telomerase subunit) could be reversed upon telomerase reactivation even in aged mice (Jaskelioff et al., 2011).

In the context of wild-type mice, introducing an additional copy of the telomerase gene, which is expressed in a wide range of epithelial tissues, led to an increased wound healing capacity of the skin (Gonzalez-Suarez et al., 2001). When this allele was introduced in a tumour-resistant genetic background (Sp53/Sp16/SArf) remarkable delay of aging in concert with an increased median lifespan of 40% compared to mice not expressing the telomerase transgene was observed (Tomas-Loba et al., 2008).

A virus (AAV) based telomerase gene therapy was found to be beneficial to extend health span, in the context of normal physiological aging in wild-type mice. In the study examining this benefit, adult and aged mice were subjected to AAV9-mTERT gene therapy to broadly express the catalytic subunit of mouse telomerase (mTERT). The health span of the TERT treated mice was significantly increased, and aging was decelerated, as indicated by a number of physiological parameters (glucose and insulin tolerance, osteoporosis, neuromuscular coordination, rota-rod, etc). In addition, their mean lifespan, compared to control groups, was increased by 24% and 13% in adult an old mice, respectively. A single intravenous administration of AAV9-TERT in adult mice resulted in an increase in telomere length in peripheral blood cells (Bernardes de Jesus et al., 2012).

Shortened telomeres have been associated with numerous diseases, such as Dyskeratosis congenita, Aplastic anaemia, Myelodysplastic Syndrome, and Fanconi anaemia. Given the severity of these diseases and the poor prognosis of the patients suffering from them, there is a need for novel therapies to treat diseases associated with short telomere length.

Aplastic anemia is a potentially life-threatening, rare and heterogeneous disorder of the blood in which the bone marrow cannot produce sufficiently enough new blood cells due to a marked reduction of immature hematopoietic stem (HSC) and progenitor cells (Scopes et al., 1994, Maciejewski et al., 1994). Accordingly, the main disease manifestations are pancytopenia and marrow hypoplasia which can emerge at any stage of life but are more frequent in young people (age 10-25 years) and the elderly (>60 years) (Marsh et al., 2009). Aplastic anemia can be acquired or inherited. The acquired type is mainly autoimmune-mediated but can also be triggered by environmental factors such as radiation, toxin and virus exposure (Nakao, 1997). The congenital form is rarer, however, mutations in more than 30 genes with functions in DNA repair, ribosome biogenesis and telomere maintenance pathways have been identified to date (Dokal & Vulliamy, 2010). A frequently observed clinical feature of aplastic anemia is short telomere length in peripheral blood leukocytes even in the absence of mutations in the telomere maintenance machinery.

Telomeres, the termini of vertebrate chromosomes are highly specialised nucleoprotein structures composed of hexanucleotide (TTAGGG) tandem repeat sequences which are bound by a six protein complex (TRF1, TRF2, TIN2, RAP1, TPP1 and POT1) termed shelterin (Blackburn, 2001, de Lange, 2005). These structures are essential for chromosome integrity by preventing telomere fusions and telomere fragility. Telomere length is controlled by the ribonucleoprotein enzyme telomerase which can de novo add telomeric sequences onto telomeres. Because telomeric sequence is naturally lost upon every cell division (known as the end replication problem) and somatic cells express telomerase at very low levels or not at all telomeres shorten throughout life. When telomeres become critically short they lose their protective function and a persistent DNA damage response at the telomeres is triggered which subsequently leads to a cellular senescence response (Harley et al., 1990, Flores et al., 2008). HSCs, in contrast to most somatic cells, show low level of telomerase activity. However, this activity is insufficient to stop telomere attrition and consequently the regeneration potential of HSCs cells may become limited during the aging process (Hiyama & Hiyama, 2007). In line with this, recipients of bone marrow transplants have shorter telomere lengths than their donors suggesting that telomerase cannot cope with increased replicative proliferation demand during the engraftment phase (Wynn et al., 1998). Moreover, telomeres have been shown to shorten much faster in patients with aplastic anemia compared to the normal aging-related attrition found in healthy individuals potentially owed to a higher than normal number of cell divisions (Ball et al., 1998).

Accelerated telomere shortening due to defects in telomere components or telomerase itself prematurely limits the proliferation potential of cells which particularly affects the tissue renewal capacity in stem cell compartments (Harley et al., 1990, Flores et al., 2005). Thus, tissues with a high proliferative index such as the hematopoietic system are particularly affected by lower than normal telomerase levels which can ultimately lead to severe disorders such as aplastic anemia (Vulliamy et al., 2002). For instance, the telomeropathy dyskeratosis congenita has been linked to mutations in 7 genes with important functions in telomere maintenance (TERT, TERC, DKC1, TIN2, NOP10, NHP2 and TCAB1) and is characterized by very short telomeres. Dyskeratosis congenita is a multisystem syndrome comprising diverse clinical features such as nail dystrophy, oral leucoplakia, abnormal skin pigmentation and cerebellar hypoplasia (Dokal, 2011). The most severe complication, however, is the development of aplastic anemia in 80% of the cases underlining that the clinical features are caused by excessive telomere shortening which eventually leads to the exhaustion of the stem cell reserve (Dokal & Vulliamy, 2010).

The causality between proliferation potential and telomere length suggests that a therapeutic intervention with telomerase, aimed at preventing telomere loss beyond a critically short length, may be a feasible strategy to treat those forms of aplastic anemia associated with limited blood forming capacity due to the presence of short telomeres. In this regard, we previously developed a telomerase (Tert) gene therapy using adeno-associated virus (AAV9) vectors. Interestingly, telomerase gene therapy using AAV9 Tert in adult wilt-type mice attenuated or reverted the aging-associated telomere erosion in peripheral blood monocytes (Bernardes de Jesus et al., 2012), suggesting that this gene therapy may be effective in the treatment of hematological disorders related to short telomeres.

To test this hypothesis we used our recently generated mouse model of aplastic anemia which recapitulates the bone marrow phenotype observed in patients (Beier et al., 2012). In this mouse model bone marrow specific depletion of the shelterin gene Trf1 cause severe telomere uncapping and provokes a DNA damage response which in turn leads to a fast clearance of those HSCs and progenitor cells deficient for Trf1. However, in this model we induce Trf1 deletion at a frequency that does not target 100% of the HSCs and progenitor cells. Therefore, cells that retain intact Trf1 undergo additional rounds of compensatory proliferation leading to fast telomere attrition. Thus, partial depletion of the stem and progenitor cell compartment by Trf1 deletion recapitulates the compensatory hyperproliferation observed after bone marrow transplantation or in autoimmune-mediated aplastic anemia, as well as presence of very short telomeres in patients owing to mutations in telomere maintenance genes. Interestingly, in our mouse model we can adjust the rate of telomere shortening through the frequency of Trf1 deletion-mediated HSC depletion which allows to control the onset of bone marrow aplasia and pancytopenia (Beier et al., 2012).

In this study we employ this mouse model of aplastic anemia to investigate whether telomerase activation using state of the art gene therapy vectors can be an effective treatment to attenuate telomere attrition and HSC depletion, and thus prevent bone marrow failure.

### SUMMARY

The invention provides compositions and methods useful for the treatment and prevention of conditions associated with short telomere length.

One aspect of the invention provides a method of treating a patient with a condition associated with short telomere length comprising administering to the patient a nucleic acid vector comprising a coding sequence for telomerase reverse transcriptase (TERT). In one embodiment, the TERT is encoded by a nucleic acid sequence comprising a sequence that is at least 90% identical to the sequence of SEQ ID NO: 1 or SEQ ID NO: 3. In one embodiment, the TERT is encoded by a nucleic acid sequence comprising the sequence of SEQ ID NO: 1 or SEQ ID NO: 3. In one embodiment, the TERT is encoded by a nucleic acid sequence consisting of the sequence of SEQ ID NO: 1 or SEQ ID NO: 3 In one embodiment, the TERT comprises an amino acid sequence that is at least 90% identical to the amino acid sequence of SEQ ID NO:2 or SEQ ID NO: 4. In one embodiment, the TERT comprises an amino acid sequence of SEQ ID NO:2 or SEQ ID NO: 4. In one embodiment, the TERT consists of the amino acid sequence of SEQ ID NO:2 or SEQ ID NO: 4. In one embodiment, the nucleic acid sequence encoding TERT is operably linked to a regulatory sequence that drives the expression of the coding sequence. In one embodiment, the vector is a non-integrative vector, such as an adeno-associated virus-based non-integrative vector. In one embodiment, the vector is an adeno-associated virus-based vector derived from a serotype 9 adeno-associated virus (AAV9). In one embodiment, the capsid of the adeno-associated virus-based vector is made of capsid proteins of the serotype 9 adeno-associated virus (AAV9), and the nucleic acid sequence contained in the capsid is flanked at both ends by internal terminal repeats corresponding to serotype 2 adenoassociated viruses. In one embodiment, the nucleic acid contained in the capsid comprises a fragment which encodes the amino acid sequence coding for TERT. In one embodiment, the vector comprises a regulatory sequence which is a constitutive promoter. In one embodiment, the regulatory sequence is the cytomegalovirus (CMV) promoter. In one embodiment, the condition associated with short telomere length is characterized by mutations in a gene or genes involved in telomere maintenance. In one embodiment, the condition associated with short telomere length is selected from the group consisting of Dyskeratosis congenita, Aplastic anaemia, Myelodysplastic Syndrome, Fanconi anaemia.

In yet further embodiments, the invention is directed to the following set of subject matters:
1. A method of treating a patient with a condition associated with short telomere length comprising administering to the patient a nucleic acid vector comprising a coding sequence for telomerase reverse transcriptase (TERT).
2. The method of 1, wherein TERT is encoded by a nucleic acid sequence comprising a sequence that is at least 90% identical to the sequence of SEQ ID NO: 1 or SEQ ID NO: 3.
3. The method of 1 or 2, wherein TERT is encoded by a nucleic acid sequence comprising the sequence of SEQ ID NO: 1 or SEQ ID NO: 3.
4. The method of any of 1-3, wherein TERT is encoded by a nucleic acid sequence consisting of the sequence of SEQ ID NO: 1 or SEQ ID NO: 3
5. The method of any of 1-4, wherein TERT comprises an amino acid sequence that is at least 90% identical to the amino acid sequence of SEQ ID NO:2 or SEQ ID NO: 4.
6. The method of any of 1-5, wherein TERT comprises an amino acid sequence of SEQ ID NO:2 or SEQ ID NO: 4.
7. The method of any of 1-6, wherein TERT consists of the amino acid sequence of SEQ ID NO:2 or SEQ ID NO: 4.
8. The method of any of 1-7, wherein the nucleic acid sequence encoding TERT is operably linked to a regulatory sequence that drives the expression of the coding sequence.
9. The method of any of 1-8, wherein the vector is a non-integrative vector.
10. The method of any of 1-9, wherein the vector is an adeno-associated virus-based non-integrative vector.
11. The method of any of 1-10, wherein the vector is an adeno-associated virus-based vector derived from a serotype 9 adeno-associated virus (AAV9).
12. The method of 11, wherein the capsid of the adeno-associated virus-based vector is made of capsid proteins of the serotype 9 adeno-associated virus (AAV9), and the nucleic acid sequence contained in the capsid is flanked at both ends by internal terminal repeats corresponding to serotype 2 adenoassociated viruses.
13. The method of 12, wherein the nucleic acid contained in the capsid comprises a fragment which encodes the amino acid sequence coding for TERT.
14. The method of any of 1-13, wherein the vector comprises a regulatory sequence which is a constitutive promoter.
15. The method of 14, wherein the regulatory sequence is the cytomegalovirus (CMV) promoter.
16. The method of any of 1-15, wherein the condition associated with short telomere length is characterized by mutations in a gene or genes involved in telomere maintenance.
17. The method of any of 1-16, wherein the condition associated with short telomere length is selected from the group consisting of Dyskeratosis congenita, Aplastic anaemia, Myelodysplastic Syndrome, Fanconi anaemia, and pulmonary fibrosis.
18. A nucleic acid vector comprising a coding sequence for telomerase reverse transcriptase (TERT) for use in treating a condition associated with short telomere length.
19. The nucleic acid vector of 18, wherein TERT is encoded by a nucleic acid sequence comprising a sequence that is at least 90% identical to the sequence of SEQ ID NO: 1 or SEQ ID NO: 3.
20. The nucleic acid vector of 18 or 19, wherein TERT is encoded by a nucleic acid sequence comprising the sequence of SEQ ID NO: 1 or SEQ ID NO: 3.
21. The nucleic acid vector of any of 18-20, wherein TERT is encoded by a nucleic acid sequence consisting of the sequence of SEQ ID NO: 1 or SEQ ID NO: 3
22. The nucleic acid vector of any of 18-21, wherein TERT comprises an amino acid sequence that is at least 90% identical to the amino acid sequence of SEQ ID NO:2 or SEQ ID NO: 4.
23. The nucleic acid vector of any of 18-22, wherein TERT comprises an amino acid sequence of SEQ ID NO:2 or SEQ ID NO: 4.
24. The nucleic acid vector of any of 18-23, wherein TERT consists of the amino acid sequence of SEQ ID NO:2 or SEQ ID NO: 4.
25. The nucleic acid vector of any of 18-24, wherein the nucleic acid sequence encoding TERT is operably linked to a regulatory sequence that drives the expression of the coding sequence.
26. The nucleic acid vector of any of 18-25, wherein the vector is a non-integrative vector.
27. The nucleic acid vector of any of 18-26, wherein the vector is an adeno-associated virus-based non-integrative vector.
28. The nucleic acid vector of any of 18-27, wherein the vector is an adeno-associated virus-based vector derived from a serotype 9 adeno-associated virus (AAV9).
29. The nucleic acid vector of 28, wherein the capsid of the adeno-associated virus-based vector is made of capsid proteins of the serotype 9 adeno-associated virus (AAV9), and the nucleic acid sequence contained in the capsid is flanked at both ends by internal terminal repeats corresponding to serotype 2 adenoassociated viruses.
30. The nucleic acid vector of 29, wherein the nucleic acid contained in the capsid comprises a fragment which encodes the amino acid sequence coding for TERT.
31. The nucleic acid vector of any of 18-30, wherein the vector comprises a regulatory sequence which is a constitutive promoter.
32. The nucleic acid vector of 31, wherein the regulatory sequence is the cytomegalovirus (CMV) promoter.
33. The nucleic acid vector of any of 18-32, wherein the condition associated with short telomere length is characterized by mutations in a gene or genes involved in telomere maintenance.
34. The nucleic acid vector of any of 18-33, wherein the condition associated with short telomere length is selected from the group consisting of Dyskeratosis congenita, Aplastic anaemia, Myelodysplastic Syndrome, Fanconi anaemia, and pulmonary fibrosis.
35. The nucleic acid vector of any of 18-34, wherein the condition associated with short telomere length is dyskeratosis congenita.

### BRIEF DESCRIPTION OF THE FIGURES

FIG. 1A-E: AAV9-Tert effects on survival (A-C) and blood counts (D-E)

FIG. 2A-E: AAV9-Tert effects on telomere length in peripheral blood (A-C) and bone marrow (D-E)

### DETAILED DESCRIPTION OF EMBODIMENTS OF THE INVENTION

The invention provides compositions and methods useful for the treatment and prevention of conditions associated with short telomere length.

A “condition associated with short telomere length” is one which is characterized by an accumulation of critically short telomeres. In certain embodiments, subjects suffering from such a condition exhibit premature onset of pathologies resulting from a defective regenerative capacity of tissues.

In certain embodiments, the condition associated with short telomere length is characterized by mutations in a gene or genes involved in telomere maintenance. Specific examples of such genetically based conditions include, but are not limited to Dyskeratosis congenita, Aplastic anaemia, Myelodysplastic Syndrome, Fanconi anaemia, and pulmonary fibrosis.

Dyskeratosis congenita (DKC) is a genetically heterogeneous human disease, which is paradigmatic of premature ageing syndromes (Dokal, 2011). DKC is characterised by the presence of short/dysfunctional telomeres owing to mutations in genes related to telomere maintenance, being the most frequently mutated those encoding proteins of the telomerase complex (i.e. TERT, TERC, NOP10, DKC1, NHP2) (Dokal, 2011; Dokal and Vulliamy, 2010; Mason and Bessler, 2011; Savage and Alter, 2008). In addition, a subset of patients carry mutations in the gene encoding TIN2 (TRF1-interacting protein) a component of the shelterin complex, which binds and protects mammalian telomeres (Dokal, 2011; Martinez and Blasco, 2011; Walne et al., 2008). Both a functional telomerase complex and a proper telomere capping structure by the shelterin proteins are required for maintenance and capping of chromosome ends, respectively.

Clinical features of patients suffering from DKC include skin abnormalities (i.e. skin hyperpigmentation), signs of premature aging (i.e. hair greying, nail dystrophy, oral leucoplakia, etc), predisposition to cancer, and several other life-threatening conditions, including aplastic anemia and pulmonary fibrosis (Armanios and Blackburn, 2012). In particular, tissues with a high proliferative index are most affected due to the loss of telomeric DNA that occurs upon each cell division. This explains why DKC patients are particularly vulnerable to impaired bone marrow function leading to pancytopenia and eventually bone marrow failure (BMF) (Armanios and Blackburn, 2012; Blasco, 2007)

Aplastic anaemia, is a life threatening bone marrow disorder characterised by hypocellular bone marrow and low blood cell counts. Patients with acquired aplastic anaemia present with leukocytes which have considerably shorter telomeres than age-matched healthy individuals (Carroll and Ly, 2009). Aplastic anaemia is frequently caused by an autoimmune mediated attack against hematopoietic stem cells. However, recent studies demonstrated that mutations in the core telomerase components TERT and TERC are the underlying cause in a clinically relevant subpopulation (Yamaguchi et al., 2003; Yamaguchi et al., 2005). Mutations in the core telomerase components TERT and TERC, as well as in the shelterin component TIN2 have been linked to this disease (Savage et al., 2006).

Myelodysplastic Syndrome (MDS) encompasses several bone marrow diseases characterised by ineffective production of the myeloid class of blood cells. Caused by progressive bone marrow failure, similar to DKC, MDS patients often report with severe anaemia and cytopenias. In approximately one third of the cases the disease progresses quickly and transforms into acute myelogenous leukemia (AML) which is particularly resistant to treatment. Even though shortened telomeres in patients with MDS suggest that insufficient or hampered telomeric maintenance is causative for the syndrome, only 3 out of 210 cases showed heterozygous TERC mutations in a previous study (Yamaguchi et al., 2003). However, a recently published study clearly circumstantiated the connection between human telomerase mutations and MDS, aplastic anaemia and AML. (Holme et al., 2012) reported various families with mutations of the telomerase components TERC and TERT which e.g. the grandfather suffered from AML, the daughter from MDS and the grandson from aplastic anaemia (Holme et al., 2012) emphasising the close relation of different clinical manifestations with impaired telomere maintenance.

Fanconi anaemia (FA) is a heterogeneous genetic disease caused by mutations in genes involved in DNA repair. Affected individuals display multiple congenital defects and haematological deficiencies at a young age of onset (Kee and D'Andrea, 2012). Manifestations related to the latter however are the predominant symptoms of this syndrome and as the disease progresses can develop into the aforementioned syndromes including aplastic anaemia, MDS and AML. Importantly, patients suffering from FA have been also shown to present shorter telomeres than normal (Gadalla et al., 2010). The facts that mutations causing FA show impaired DNA damage response (DDR) and telomeres are particularly vulnerable to replicative stress may provide an explanation for the observed telomere erosion. In support of this Callen et al. (2002) suggested that in FA patients increased telomere breakage in concert with replicative shortening account for the observed telomere shortening.

Pulmonary fibrosis refers to a condition characterised by scarring of the lung tissue. Pulmonary fibrosis can be caused by many factors, including chronic inflammatory processes, infections, environmental compounds, ionizing radiation (for example radiation therapy to treat tumors of the chest), chronic medical conditions (lupus, rheumatoid arthritis). Idiopathic pulmonary fibrosis (IPF) refers to pulmonary fibrosis without an identifiable cause.

Accordingly, the invention provides methods of treating a patient suffering from a condition associated with short telomere length comprising administering to the patient an agent which increases the telomere length of the patient. In one embodiment, the agent prevents degradation of the chromosomal ends. In one embodiment, the agent increases the activity of telomerase reverse transcriptase (TERT). In one embodiment, the method of treatment is a gene therapy method comprises administering to the patient a nucleic acid vector comprising a coding sequence for telomerase reverse transcriptase (TERT).

In certain embodiments, the TERT sequence used in the gene therapy vector is derived from the same species as the subject. For example, gene therapy in humans would be carried out using the human TERT sequence. Gene therapy in mice would be carried out using the mouse TERT sequence, as described in the examples. In one embodiment, the TERT is encoded by the nucleic acid sequence as set forth in SEQ ID NO: 1 or SEQ ID NO: 3 (human TERT variants 1 and 2), or is an active fragment or functional equivalent of SEQ ID NO: 1 or SEQ ID NO: 3. The polypeptide sequence encoded by SEQ ID NO: 1 is set forth in SEQ ID NO: 2. The polypeptide encoded by SEQ ID NO: 3 is set forth in SEQ ID NO: 4. As used herein, “functional equivalent” refers to a nucleic acid molecule that encodes a polypeptide that has TERT activity or a polypeptide that has TERT activity. The functional equivalent may displays 50%, 60%, 70%, 80%, 90%, 95%, 98%, 99%, 100% or more activity compared to TERT encoded by SEQ ID NO: 1 or SEQ ID NO: 3. Functional equivalents may be artificial or naturally-occurring. For example, naturally- occurring variants of the TERT sequence in a population fall within the scope of functional equivalent. TERT sequences derived from other species also fall within the scope of the term “functional equivalent”, in particular the murine TERT sequence given in SEQ ID NO: 5. In a particular embodiment, the functional equivalent is a nucleic acid with a nucleotide sequence having at least 75%, 80%>, 85%, 90%, 95%, 96%, 97%, 98%, 99%, 99.5%, 99.9% identity to SEQ ID NO: 1 or SEQ ID NO: 3. In a further embodiment, the functional equivalent is a polypeptide with an amino acid sequence having at least 75%, 80%, 85%, 90%, 95%, 96%, 97%, 98%, 99%, 99.5%, 99.9% identity to SEQ ID NO: 2 or SEQ ID NO: 4. In the case of functional equivalents, sequence identity should be calculated along the entire length of the nucleic acid. Functional equivalents may contain one or more, e.g. 2, 3, 4, 5, 10, 15, 20, 30 or more, nucleotide insertions, deletions and/or substitutions when compared to SEQ ID NO: 1 or SEQ ID NO: 3. The term “functional equivalent” also encompasses nucleic acid sequences that encode a TERT polypeptide with at least 75%, 80%, 85%, 90%, 95%, 96%, 97%, 98%, 99%, 99.5%, 99.9% sequence identity to the sequence as set forth in SEQ ID NO:2 or SEQ ID NO: 4, but that show little homology to the nucleic acid sequence given in SEQ ID NO: 1 or SEQ ID NO: 3 because of the degeneracy of the genetic code.

As used herein, the term “active fragment” refers to a nucleic acid molecule that encodes a polypeptide that has TERT activity or polypeptide that has TERT activity, but which is a fragment of the nucleic acid as set forth in SEQ ID NO: 1 or SEQ ID NO: 3 or the amino acid sequence as set forth in SEQ ID NO: 2 or SEQ ID NO: 4. An active fragment may be of any size provided that TERT activity is retained. A fragment will have at least 75%, 80%, 85%, 90%, 95%, 96%, 97%, 98%, 99%, 99.5%, 100% identity to SEQ ID NO: 1-4 along the length of the alignment between the shorter fragment and SEQ ID NO: 1-4.

Fusion proteins including these fragments can be comprised in the nucleic acid vectors needed to carry out the invention. For example, an additional 5, 10, 20, 30, 40, 50 or even 100 amino acid residues from the polypeptide sequence, or from a homologous sequence, may be included at either or both the C terminal and/or N terminus without prejudicing the ability of the polypeptide fragment to fold correctly and exhibit biological activity.

Sequence identity may be calculated by any one of the various methods in the art, including for example BLAST (Altschul S F, Gish W, Miller W, Myers E W, Lipman D J (1990). “Basic local alignment search tool”. J Mol Biol 215 (3): 403-410) and FASTA (Lipman, D J; Pearson, W R (1985). “Rapid and sensitive protein similarity searches”. Science 227 (4693): 1435-41; http://fasta.bioch.Virginia,edu/fasta_www2/fasta_list2.shtml) and variations on these alignment programs

In one embodiment, the method of treatment is a gene therapy method and/or the nucleic acid vector used is a gene therapy vector. Gene therapy methods and vectors are well known in the art and generally comprise delivering a nucleic acid encoding a therapeutically active protein to a subject. The nucleic acid may be delivered in a number of ways including delivering naked DNA such as plasmid or mini-circles, the use of liposomes or cationic polymers or other engineered nano-particles containing the nucleic acid, or viral vectors that encapsidate the nucleic acid.

In a further embodiment, the gene therapy is achieved using stable transformation of organisms with an inducible expression system. Suitable inducible expression systems are known in the art and include the CRE-LOX recombinase based system which is suitable for use in mice and tetracycline-regulated which can be used in the treatment of human subjects.

In one embodiment the gene therapy vector is a viral vector. Viral gene therapy vectors are well known in the art. Vectors include integrative and non-integrative vectors such as those based on retroviruses, adenoviruses (AdV), adeno- associated viruses (AAV), lentiviruses, pox viruses, alphaviruses, and herpes viruses.

Using non-integrative viral vectors, such as AAV, seems to be particularly advantageous. In one aspect, this is because non-integrative vectors do not cause any permanent genetic modification. Second, the vectors target to adult tissues, avoiding having the subjects under the effect of constitutive telomerase expression from early stages of development. Additionally, non-integrative vectors effectively incorporate a safety mechanism to avoid over-proliferation of TERT expressing cells. Cells will lose the vector (and, as a consequence, the telomerase expression) if they start proliferating quickly.

Particular examples of suitable non-integrative vectors include those based on adenoviruses (AdV) in particular gutless adenoviruses, adeno-associated viruses (AAV), integrase deficient lentiviruses , pox viruses, alphaviruses, and herpes viruses. Preferably, the non-integrative vector used in the invention is an adeno-associated virus-based non-integrative vector, similar to natural adeno-associated virus particles. AAV preferentially targets post-mitotic tissues, which are considered more resistant to cancer than the highly proliferative ones. Examples of adeno-associated virus-based non integrative vectors include vectors based on any AAV serotype, i.e. AAV1, AAV2, AAV3, AAV4, AAV5, AAV6, AAV7, AAV8, AAV9, AAV10, AAV11 and pseudotyped AAV. Tissue specificity is determined by the capsid serotype. Pseudotyping of AAV vectors and capsid engineering to alter their tropism range will likely be important to their use in therapy.

Vectors derived from adeno-associated viruses (AAVs) have emerged as one of the vectors of choice for many gene transfer applications because of their many desirable properties, including capability to transduce a broad range of tissues at high efficiency, poor immunogenicity and an excellent safety profile (Merten, Geny-Fiamma et al. 2005; Buning, Perabo et al. 2008), toxicity being absent in many preclinical models (Niemeyer, Herzog et al Blood 2009; Mas, Montane et al Diabetes 2006; Jiang, Lillicrap et al blood 2006; Ghosh, Yue et al Molecular therapy 2007; Tafuro, Ayuso et al cardiovascular research 2009). AAV vectors transduce post-mitotic cells and can sustain long-term gene expression (up to several years) both in small and large animal models of disease (Niemeyer, Herzog et al Blood 2009; Mas, Montane et al Diabetes 2006; Jiang, Lillicrap et al blood 2006; Ghosh, Yue et al Molecular therapy 2007; Tafuro, Ayuso et al cardiovascular research 2009). Safety and efficacy of AAV gene transfer has been extensively studied in humans with encouraging results in the liver, muscle, CNS, and retina (Manno et al Nat medicine 2006, Stroes et al ATVB 2008, Kaplitt, Feigin, Lancet 2009; Maguire, Simonelli et al NEJM 2008; Bainbridge et al NEJM 2008).

AAV2 is the best characterized serotype for gene transfer studies both in humans and experimental models. AAV2 presents natural tropism towards skeletal muscles, neurons, vascular smooth muscle cells and hepatocytes. AAV2 is therefore a good choice of vector to target these tissues, in particular when using the methods or vectors of the invention to treat a condition associated with one of these tissues. For example, treatment of neuromuscular degeneration may be targeted to skeletal muscle and/or neurons in this way.

Newly isolated serotypes, such as AAV7, AAV8, and AAV9 have been successfully adopted in preclinical studies (Gao, Alvira et al PNAS 2002). Although limited immunologic responses have been detected in human subjects treated with AAV2 or AAV1 against the AAV capsid (Manno et al Nat Med 2006; Mingozzi et al Nat Med 2007; Brantly et al PNAS 2009; Mingozzi et al blood 2009), long term expression of the therapeutic gene is possible depending on the target tissue and the route of administration (Brantly et al PNAS 2009; Simonelli et al mol therapy 2010). In addition, the use of non-human serotypes, like AAV8 and AAV9, might be useful to overcome these immunological responses in subjects, and clinical trials have just commenced (ClinicalTrials.gov Identifier: NCT00979238). Altogether, these encouraging data suggest that AAV vectors are useful tools to treat human diseases with a high safety and efficient profile.

The choice of adeno-associated viruses of wide tropism, such as those derived from serotype 9 adeno-associated virus (AAV9) is particularly advantageous when treating conditions associated with short telomere length. AAV9 viruses have shown efficient transduction in a broad range of tissues, with high tropism for liver, heart and skeletal muscle (Inagaki et al Molecular Therapy 2006) and thus the beneficial effects of gene therapy can be achieved in more tissues. In addition, AAV9 vectors have the unique ability to cross the blood-brain-barrier and target the brain upon intravenous injection in adult mice and cats (Foust et al Nature biotechnology 2009; Duque et al Molecular therapy et al 2009).

One aspect of the invention provides a system in which the capsid (which is the part of the virus which determines the virus tropism) of the adeno-associated virus- based vector is made of capsid proteins of the serotype 9 adeno-associated virus (AAV9). In one embodiment of the viral vectors for use in the invention, the polynucleotide sequence packed in the capsid is flanked by internal terminal repeats (ITRs) of an adeno-associated virus, preferably of serotype 2 which has been extensively characterised in the art, and presents a coding sequence located between the ITRs. As set out above, the nucleic acid preferably codes for a functional TERT polypeptide. In one embodiment, the regulatory sequence operatively linked to the TERT coding sequence is the cytomegalovirus promoter (CMV), although other suitable regulatory sequences will be known to those of skill in the art.

When treating conditions associated with short telomere length, it is advantageous to target the treatment to the effected tissues. The choice of AAV serotype for the capsid protein of the gene therapy vector may be thus based on the desired site of gene therapy. If the target tissue is skeletal muscle, for example, in treating loss of neuromuscular coordination, AAV1- and AAV6-based viral vectors can be used. Both of these serotypes are more efficient at trans fecting muscle than other AAV serotypes. AAV3 is useful for trans fecting haematopoietic cells. A thorough review of AAV-based vectors for gene therapy can be found in Shi et al, (2008) “AAV-based targeting gene therapy” Am. J. Immunol. 4:51-65.

Alternatively, other viral vectors can be used in the present invention. Any vector compatible with use in gene therapy can be used in the present invention. Heilbronn & Weger (2010) Handb Exp Pharmacol. 197: 143-70 provides a review of viral vectors that are useful in gene therapy. In accordance with all the previous discussion, vectors comprising a coding sequence for telomerase reverse transcriptase (TERT) suitable for use in gene therapy are an important point for putting the invention into practice. Suitable gene therapy vectors include any kind of particle that comprises a polynucleotide fragment encoding the telomerase reverse transcriptase (TERT) protein, operably linked to a regulatory element such as a promoter, which allows the expression of a functional TERT protein demonstrating telomerase reverse transcriptase activity in the targeted cells. Preferably, TERT is encoded by the nucleic acid sequence as set forth in SEQ ID NO: 1 or SEQ ID NO: 3, or is an active fragment or functional equivalent of TERT.

The term gene therapy vector includes within its scope naked DNA molecules such as plasmids or mini-circles, i.e. circular DNA molecules which do not contain bacterial DNA sequences, provided that the TERT coding sequence and its linked regulatory element are inserted in the plasmid, as well as to more complicated systems, such as particles with the structure of virions (viral particles), comprising at least a capsid and at least a polynucleotide sequence, with a size that allows the polynucleotide sequence to be packed within the capsid in a manner similar to that of the native genome of the virus of origin of the capsid. The polynucleotide sequence must include a region where the TERT coding sequence and its linked regulatory element are inserted such that the telomerase reverse transcriptase protein can be expressed from that polynucleotide sequence once the viral particle has infected a cell.

In one embodiment, the gene therapy vector suitable for being used in the invention is a non-integrative vector, such as an adeno-associated virus-based non-integrative vector. For the purposes of the invention, the choice of non-integrative vectors seems to be particularly advantageous, because they do not cause any permanent genetic modification. Also, as stated before, such vectors incorporate a safety mechanism to avoid over-proliferation of TERT expressing cells that will lose the vector if the cells start proliferating quickly.

Adeno-associated virus-based vectors derived from a serotype 9 adeno-associated virus (AAV9) are preferred because the beneficial effects can be achieved in more tissues (see above). In one particularly preferred embodiment, the regulatory sequence operatively linked to the TERT coding sequence is the cytomegalovirus promoter (CMV). The nucleic acid sequence encoding TERT is operably linked to a regulatory sequence that drives the expression of the coding sequence. As used herein, the term “regulatory element” means a nucleic acid sequence that serves as a promoter, i.e., regulates expression of a nucleic acid sequence operably linked to the promoter. Such “regulatory elements” or “promoters” can control the expression of linked nucleic acid sequences either constitutively or inducible.

The regulatory sequence may be a constitutive promoter. An example of a regulatory sequence which is a constitutive promoter is the cytomegalovirus (CMV) promoter.

The expression of TERT following gene therapy according to the invention persists for a time of several months to several years. In mice, TERT expression was detectable after 5 months. In monkey, gene expression following gene therapy with an AAV-based vector has been detected up to 6 years after treatment and up to 8 years in dogs (Rivera et al Blood 2005, and Niemeyer et al blood 2009). Frequent repetition of treatment using the methods and vectors of the invention is therefore not necessary. In one embodiment of the invention, the subject is treated once. In an alternative embodiment, the subject is treated initially, and is then treated again once TERT expression levels decrease by about 50% of those attained immediately following treatment. Treatment may be repeated with the same or alternative vector to maintain the reduction in age-related disorders if necessary, for example annually, or once every 5 years or once a decade. When administering a second or subsequent dose, it may be necessary to use a different gene therapy vector, for example when using an AAV-based vector the second and subsequent administrations may be a vector with a capsid derived from a different serotype than that used for the first administration. It is possible that a subject may develop neutralising antibodies to the first gene therapy vector, making it ineffective if administered a second or subsequent time (Amado et al (2010) Science Translational Medicine 2(21):21ral6).

The methods of treatment of the invention have the effect of treating and/or preventing conditions associated with short telomere length. In a further aspect, therefore, the invention refers to a gene therapy method or the use of a nucleic acid vector as described above, for use in the treatment or prevention in a subject of condition associated with short telomere length, including but not limited to genetically based conditions such as Dyskeratosis congenita, Aplastic anaemia, Myelodysplastic Syndrome, Fanconi anaemia, and pulmonary fibrosis.

The effectiveness of treatment of the conditions associated with short telomere length can be measured by various methods known in the art. In one embodiment, the effectiveness of the treatment is measured by an increase in lifespan of a treated patient suffering from a condition associated with short telomere length as compared to the expected lifespan of an untreated patient suffering from the same condition. In certain embodiments, the lifespan is extended by 5%, 10%, 15%, 20% or more, with reference to the expected lifespan for a patient suffering from the same condition.

In one embodiment, the effectiveness of the treatment is measured by a delayed or prevented bone marrow failure in a treated patient suffering from a condition associated with short telomere as compared to the expected onset of bone marrow failure in an untreated patient suffering from the same condition. In certain embodiments, the delay in the onset of bone marrow failure of a treated patient suffering from a condition associated with short telomere length is extended by 5%, 10%, 15%, 20% or more, with reference to the expected onset of bone marrow failure for an untreated patient suffering from the same condition.

In one embodiment, the effectiveness of the treatment is measured by an increase in overall fitness of a treated patient suffering from a condition associated with short telomere length treated as compared to the overall fitness of an untreated patient suffering from the same condition. Overall fitness can be determined by measuring physical attributes associated with the particular condition. Examples of such physical attributes include skin abnormalities (such as skin hyperpigmentation), premature aging (such as hair greying, nail dystrophy, oral leucoplakia), and anaemic pallor. Dokal, I. 2011. Hematology Am Soc Hematol Educ Program, 480-486. Thus an increase in overall fitness can be determined by a decrease in physical attributes associated with the particular condition exhibited by the treated patient. Overall fitness can also be measure by determining the blood count of the patient. In one embodiment, increased overall fitness is measured by determining the amount of leukocytes, lymphocytes, thrombocytes in a peripheral blood sample. Higher blood count indicates an increased overall fitness. In certain embodiments, the blood count in a treated patient is increased by 5%, 10%, 15%, 20% or more, with reference to the blood count of an untreated patient suffering from the same condition.

The efficacy of the treatment can also be measured by directly determining telomere length in sample taken from the patient. Telomere length can be measured, for example, by using standard hybridization techniques, such as fluorescence in situ hybridization (FISH), Quantitative Fluorescent in situ hybridization (Q-FISH), or High Throughput Quantitative Fluorescent in situ hybridization (HT Q-FISH). (Gonzalez-Suarez, Samper et al. 2001) in a sample taken from the patient, Samples suitable for telomere analysis include bone marrow tissue and blood samples. Telomere length can also be measured as described in Slagboom et al or Canela et al. (2007, PNAS 104:5300-5305).

In a particular embodiment, samples are taken from the patient undergoing treatment throughout the course of the treatment so that both absolute telomere length and the rate of telomere shortening over the course of treatment can be determined. Samples may be taken every day during the course of treatment, or at longer intervals. In one embodiment, samples are taken once a week, once every two week, once every three weeks, once every 4 weeks, once every five weeks, once every six weeks or longer.

Comparison of telomere length can be measured by a comparing the proportion of short telomeres in a sample taken from a patient. In one embodiment, the proportion of short telomeres is the fraction of telomeres presenting an intensity below the mean intensity of the sample as measured by a in situ hybridization technique, such as FISH or Q-FISH. In embodiment, the proportion of short telomeres is the fraction of telomeres presenting an intensity 75%, 70%, 65%, 60%, 55%, 50%, 45%, 40% or more below the mean intensity of the sample. In one particular embodiment, the proportion of short telomeres is the fraction of telomeres presenting an intensity 50% or more below the mean intensity of the sample.

In another embodiments, the proportion of short telomeres is the fraction of telomeres below a certain length, e.g. 8 kb, 7 kb, 6 kb, 5 kb, or shorter In one embodiment, the proportion of short telomeres is the fraction of telomeres 8 kb or shorter . In another embodiment, the proportion of short telomeres is the fraction of telomeres 7 kb or shorter. In another embodiment, the proportion of short telomeres is the fraction of telomeres 6 kb or shorter. In another embodiment, the proportion of short telomeres is the fraction of telomeres 5 kb or shorter. In another embodiment, the proportion of short telomeres is the fraction of telomeres 4 kb or shorter. In another embodiment, the proportion of short telomeres is the fraction of telomeres 3 kb or shorter.

In one embodiment, the effectiveness of the treatment is measured by a decrease in the proportion of short telomeres in sample taken from a treated patient suffering from a condition associated with short telomere length as compared to a control sample. In one embodiment, the proportion of short telomeres in a sample taken from a treated patient is decreased by 10%, 20%, 30%, 40%, 50%, 60%, 70%, or greater as compared to a control sample. In one embodiment, the control sample is a sample taken from the same patient prior to the treatment, or taken at an earlier stage of the treatment. In another embodiment, the control sample is a sample taken from a patient suffering from the same condition and not provided the treatment.

In a further aspect, the invention is applied to the subject by administering a pharmaceutical composition comprising an effective amount of any one of the gene therapy vectors compatible with the invention described above.

A “pharmaceutical composition” is intended to include the combination of an active agent with a carrier, inert or active, making the composition suitable for diagnostic or therapeutic use in vitro, in vivo or ex vivo.

“Composition” is intended to mean a combination of active agent and another compound or composition, inert (for example, a detectable agent or label) or active. An “effective amount” is an amount sufficient to effect beneficial or desired results. An effective amount can be administered in one or more administrations, applications or dosages.

They will usually include components in addition to the active component (such as the gene therapy vector) e.g. they typically include one or more pharmaceutical carrier(s) and/or excipient(s). A thorough discussion of such components is available in Gennaro (2000) Remington: The Science and Practice of Pharmacy. 20th edition, ISBN: 0683306472.

Compositions will generally be administered to a subject in aqueous form. Prior to administration, however, the composition may have been in a non-aqueous form. For instance, although some viral vectors are manufactured in aqueous form, then filled and distributed and administered also in aqueous form, other viral vectors are lyophilised during manufacture and are reconstituted into an aqueous form at the time of use. Thus a composition of the invention may be dried, such as a lyophilised formulation. The composition may include preservatives such as thiomersal or 2-phenoxyethanol. It is preferred, however, that the composition should be substantially free from (i.e. less than 5 μg/m\acute{ï}) mercurial material e.g. thiomersal-free.

To control tonicity, it is preferred to include a physiological salt, such as a sodium salt. Sodium chloride (NaCl) is preferred, which may be present at between 1 and 20 mg/ml e.g. about 10+2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The composition may include material for a single administration, or may include material for multiple administrations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Compositions of the invention for use in humans are typically administered in a dosage volume of about 0.5 ml, although a half dose (i.e. about 0.25 ml) may be administered to children.

As well as methods of treatment described herein, the invention also provides a nucleic acid sequence encoding a TERT for use in therapy. The invention also provides a nucleic acid vector comprising a coding sequence for telomerase reverse transcriptase (TERT), for use in a method of therapy and a gene therapy vector comprising a coding sequence for telomerase reverse transcriptase (TERT), for use in a method of therapy. In particular, the therapy may be treating or preventing a condition associated with short telomere length. As described for methods of treatment, the TERT nucleic acid sequence may be the sequence as recited in SEQ ID NO: 1 or SEQ ID NO: 3 or a fragment or functional equivalent thereof. The TERT protein may have a sequence as recited in SEQ ID NO: 2 or SEQ ID NO: 4, or a fragment or functional equivalent thereof.

The term “patient” refers to a mammal. In certain embodiments the patient is a rodent, primate, ungulate, cat, dog, or other domestic pet or domesticated mammal In certain embodiments, the mammal is a mouse, rat, rabbit, pig, horse, sheep, cow, domestic cat or dog, or a human. In a preferred embodiment, the patient is a human.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### EXAMPLES

### Example 1 Mouse Model for Dyskeratosis Congenita

Mice of C57B6 background that are homozygous carrier of a conditional TRF1 transgene (TRF1^{flox/flox}) and further are transgenic for the Cre-recombinase under the control of the endogenous and interferon-inducible Mx1 promoter will be used to test the efficacy of the telomerase gene therapy to treat Dyskeratosis congenital (DKC). To exclusively study the effects of TRF1 ablation in the hematopoietic compartment bone marrow will be transplanted from these mice into irradiated wild-type mice as described previously (Beier et al., 2012). One month after transplantation the mice will be injected via their tail vein with 4×10¹² AAV9 genomes carrying the mTERT cDNA under the control of the potent cytomegalovirus promoter (for virus production, see below 3.3). By analogy, empty AAV9 lacking the telomerase gene will be injected into a control group. Furthermore, to follow the viral tropism and transgene expression over time, a separate group of animals will be injected with AAV9-eGFP. One week after the virus infection TRF1 deletion in the bone marrow will be induced by long-term polyinosinic-polycytidylic acid (pI:pC) treatment with intraperitoneal injections every third day. pI:pC acts as immunostimulant and activates Cre expression which in turn leads to TRF1 deletion in approximately 50% of hematopoietic cells (upon each injection) with the above mentioned consequences (see 2). In contrast to pI:pC treatment, animal groups previously infected with AAV9-mTERT and AAV9-empty will not undergo pI:pC treatment to serve as additional control cohorts.

With this experimental design the dramatic telomere shortening, owed to compensatory proliferation in the remaining hematopoietic cells which have not lost TRF1, by ectopic expression of telomerase, will be reduced. The strongest measure to assess the effectiveness of the gene therapy is an extended life span by virtue of delayed or prevented bone marrow failure (end point of experiment=death of animals) Furthermore, successful telomerase treatment should show improvements with regards to overall fitness of the animals, i e no skin abnormalities and no anaemic pallor. The latter goes hand in hand with higher blood counts (leukocytes, lymphocytes, thrombocytes), which will be determined from peripheral blood samples. The efficacy of telomerase expression on the molecular level will include telomere length measurements. To so Q-FISH analysis from bone marrow tissue sections and high throughput Q-FISH analysis from peripheral blood samples will be performed. For the second, blood will be taken every three to four weeks throughout the course of the experiment. In this way not only absolute telomere length, but also the rate of telomere shortening over time can be determined. Moreover, attenuated or abolished replicative senescence and exhaustion of stem and progenitor cells in the hematopoietic compartment will be monitored by assessment of common senescent markers such as beta-galactosidase activity and p21 protein levels. These markers as well as γH2AX and phospho-CHK1, molecular markers for replicative stress, can then be correlated with telomere length and survival of the animals.

### Example 2 Production of Viruses

AAV based viral vectors for transduction will be generated by triple transfection of HEK293T cells as described in (Matsushita et al., 1998). Briefly, to 80% confluence grown cells are co-transfected with plasmids (1) carrying the expression cassette flanked by the AAV9 viral ITRs, (2) a helper plasmid carrying the AAV rep2 and cap9 genes, and (3) a plasmid carrying the adenovirus helper functions. The expression cassettes harbour murine TERT under the control of CMV promoter plus 3′-UTR (AAV9-mTERT), CMV promoter (AAV9-empty) alone and eGFP under the control of CMV promoter and SV40 polyA signal (AAV9-eGFP). Vectors are purified following an optimised method based on two consecutive cesium chloride gradients (Ayuso et al., 2010). Titres of viral genomes particles are determined by quantitative real time PCR. Viruses can be stably kept at −80° C. until infection of animals.

### Example 3 Telomere Analysis

### Telomere Q-FISH Analysis on Paraffin Sections

Q-FISH determination on paraffin-embedded tissue sections mice are hybridized with a PNA-telomeric probe, and fluorescence intensity of telomeres are determined as described (Gonzalez-Suarez, Samper et al. 2001). Quantitative image analysis is performed using the Definiens Developer Cell software (version XD 1.2; Definiens AG). For statistical analysis a two-tailed Student t test is used to assess significance (GraphPad Prism software).

### Quantitative Real-Time RT-PCR

Total RNA from tissues is extracted with Trizol (Life Technologies). RNA samples are DNase I treated and are used as template for a reverse transcription reaction using random primers and Superscript Reverse Transcriptase (Life

Technologies), according to the manufacturer's guidelines. Quantitative real-time PCR is performed using an ABI

PRISM 7700 (Applied Biosystems), using DNA Master SYBR Green I mix (Applied Biosystems).

The primers:

(SEQ ID NO: 7)
Actin-For: GGCACCACACCTTCTACAATG;
(SEQ ID NO: 8)
Actin-Rev: GTGGTGGTGAAGCTGTAG;
(SEQ ID NO: 9)
TERT-For: GGATTGCCACTGGCTCCG;
(SEQ ID NO: 10)
TERT-Rev: TGCCTGACCTCCTCTTGTGAC.
(SEQ ID NO: 11)
p16-For: CGTACCCCGATTCAGGTGAT
(SEQ ID NO: 12)
p16-Rev: TTGAGCAGAAGAGCTGCTACGT
(SEQ ID NO: 13)
Axin2-For: GGCAAAGTGGAGAGGATCGAC
(SEQ ID NO: 14)
Axin2-Rev: TCGTGGCTGTTGCGTAGG
(SEQ ID NO: 15)
Cyclin D1-For: TGCGCCCTCCGTATCTTAC
(SEQ ID NO: 16)
Cyclin D1-Rev: ATCTTAGAGGCCACGAACATGC
(SEQ ID NO: 17)
CD44-For: CAGCCTACTGGAGATCAGGATGA
(SEQ ID NO: 18)
CD44-Rev: GGAGTCCTTGGATGAGTCTCGA
(SEQ ID NO: 19)
KIf4-For: GCGAACTCACACAGGCGAGAAACC
(SEQ ID NO: 20)
KIf4-Rev: TCGCTTCCTCTTCCTCCGACACA
(SEQ ID NO: 21)
Tieg1-For: CCCATTGCCCCTGCTCCTG
(SEQ ID NO: 22)
Tieg 1-Rev: TGTGTCCGCCGGTGTCTGG

Statistical analyses (Student's t-test) is performed on the Ct values as described before (Munoz, Blanco et al. 2005).

### Example 4 Telomerase Gene Therapy in Aplastic Anemia

### Mice and Animal Procedures

Mice were of pure C57/BL6 background and were produced and housed at the specific pathogen-free (SPF) animal house of the CNIO in Madrid, Spain. Trf1^{lox/lox} Mx1-Cre and Trf1^{lox/lox} Mx1-wt mice were generated previously described (Martinez et al., 2009) EN REF 20. For bone marrow transplantation 10 weeks old Ttf1^{lox/lox} Mx1-Cre mice were used as bone marrow donors for transplantation into 8 weeks old lethally (12Gy) irradiated wild-type mice as previously described (Beier et al., 2012, Samper et al., 2002). A total of 2 million cells were transplanted via tail vein injection at a donor:recipient ratio of 1:8 and mice were left for a latency period of 30 days to allow bone marrow reconstitution. To induce Cre expression, mice were intraperitoneally injected with polyinosinic-polycytidylic acid (pI:pC; Sigma-Aldrich) (15 ug/g body weight) 3 times per week for a total duration of 5 weeks. Mice were left for an additional week before they were randomly assigned to two groups for the treatment with AAV9-Tert or AAV9-empty gene therapy vectors. Vectors were administered via tail vein injection at a concentration of 4×10E12 viral genomes per mouse.

### Gene Therapy Vector Production

Viral vectors were generated as described previously (Matsushita et al., 1998) and purified described in (Ayuso et al., 2010). Briefly, vectors were produced through triple transfection of HEK293T. Cells were grown in roller bottles (Corning, NY, USA) in Dulbecco's Modified Eagle's Medium supplemented with FBS (10% v/v) to 80% confluence and then co-transfected with: plasmid-1 carrying the expression cassette for gene of interest flanked by the AAV2 viral ITRs; plasmid-2 carrying the AAV rep2 and cap9 genes; plasmid-3 carrying the adenovirus helper functions (plasmids were kindly provided by K. A. High, Children's Hospital of Philadelphia). Expression cassettes were under the control of the cytomegalovirus (CMV) promoter and contained a SV40 polyA signal for EGFP and the CMV promoter and the 3′UTR of the Tert gene as polyA signal for Tert. AAV9 particles were purified following an optimized method using two caesium chloride gradients, dialysed against PBS, filtered and stored at −80° C. until use (Ayuso et al., 2010). Viral genomes particles titres were determined by a standardized quantitative real time PCR method(Ayuso et al., 2014) and primers specific for the CMV sequence:

(SEQ ID NO: 23)
CMV-Forward: 5′-CAATTACGGGGTCATTAGTTCATAGC;
(SEQ ID NO: 24)
CMV-Reverse: 5′-ATACGTAGATGTACTGCCAAGTAGGA

### Histology

Bone marrow samples (sternum or tibia bone) were fixed in phosphate-buffered 4% formaldehyde and bones after decalcification paraffin embedded. 5 μm tissue sections were stained with Hematoxylin-Eosin for histological bone marrow assessment. Immunohistochemistry was performed on deparaffinized tissues sections. After antigen retrieval samples were processed with the anti-EGFP antibodies (rabbit anti-EGFP, 1:200; Abcam, ab290). EGFP positive cells were counted in a semi automated way using ImageJ software.

### FACS Sorting

For sorting of HSCs whole bone marrow cells were extracted from the long bones (femur & tibia) as previously described (Samper et al., 2002). Erythrocytes were lysed by incubating cells for 10 min in 10 ml erythrocyte lysis buffer (Roche), washed once with 10 ml PBS, and resuspended in FACS buffer (PBS, 2 mM EDTA, 0.3% BSA) containing Fc-block (1:400) at a concentration of 5-10×10\hat{ }6 cell/100 μl. Cells were incubated for 10 min and washed once in FACS buffer. Cells were then resuspended in FACS buffer at 20-25×10\hat{ }6 cell/ml and the antibody cocktail was added as follows: Anti-sca-1-PerCP-Cy5.5 (1:200), lin cocktail-eFluor450 (1:50) (all eBioscience), and anti-c-kit-APC-H7 (1:100) (BD Pharmingen). Cells were incubated for 30 min. After washing cells twice with PBS, 2 L of DAPI (200 g/mL) was added and cells were subsequently sorted in a FACS ARIA IIu (Becton Dickinson, San Jose, CA) into HSCs (lin negative, scal and c-kit positive) and lineage positive (lin positive) fractions.

### Colony Forming Assay

Short-term colony-forming assay (CFA) was performed by plating 1×10⁴ and 2×10⁴ freshly isolated mononucleated bone marrow cells (erythrocytes were lysed as described above) in 35-mm dishes (StemCell Technologies) containing Methocult (methylcellulose-based) media (StemCell Technologies) as described in the manufacturer's protocol. All experiments were performed in duplicates and the numbers of colonies formed were counted after 12 days incubation at 37° C.

### Blood Counts

Peripheral blood was drawn from the facial vein (˜50 μl) and collected into anti-coagulation tubes (EDTA). Blood counts were determined using an Abacus Junior Vet veterinary hematology analyzer.

### Quantitative Real-Time PCR and Western Blots

Total RNA from whole bone marrow extracts or FACS sorted bone marrow cells was isolated using Qiagen's RNeasy mini kit according to the manufacturer protocol. The optional DNasel digest was always performed. Quantitative real-time PCR was performed using an ABI PRISM 7700 or QuantStudio 6 Flex (both Applied Biosystems). Primers sequences for Tert and reference genes Act1 and TBP are as follows:

(SEQ ID NO: 9)
Tert-Forward 5′GGATTGCCACTGGCTCCG;
(SEQ ID NO: 10)
Tert-Reverse 5′TGCCTGACCTCCTCTTGTGAC;
(SEQ ID NO: 7)
Actin-Forward 5′GGCACCACACCTTCTACAATG;
(SEQ ID NO: 8)
Actin-Reverse 5′GTGGTGGTGAAGCTGTAG;
(SEQ ID NO: 25)
TBP-Forward 5′CTTCCTGCCACAATGTCACAG;
(SEQ ID NO: 26)
TBP-Reverse 5′CCTTTCTCATGCTTGCTTCTCTG

### Q-FISH Telomere Analysis

Q-FISH analysis on bone marrow tissues sections was performed as described previously (Samper et al., 2000). Briefly, tissues sections were post fixed in 4% formaldehyde for 5 min, washed 3×5 min in PBS and incubated at 37° C. for 15 min in pepsin solution (0.1% Porcine Pepsin, Sigma; 0.01M HCl, Merck). Washes and fixation was repeated and slides dehydrated in a 70%-90%-100% ethanol series (5 min each). Slides were 10 min air-dried and 30 μl of telomere probe mix added to each slide (10 mM TrisCl pH 7, 25 mM MgCl2, 9mM citric acid, 82 mM Na2HPO4, 70% deionized formamide (Sigma), 0.25% blocking reagent (Roche) and 0.5 mg/ml Telomeric PNA probe (Panagene)), a cover slip added and slides incubated for 3 min at 85° C., and 2 h at room temperature in a wet chamber in the dark. Slides were washed 2×15 min in 10 mM TrisCl pH 7, 0.1% BSA in 70% formamide under vigorous shaking, then 3×5 min in TBS 0.08% Tween20, and then incubated in a 40,6-diamidino-2-phenylindole (DAPI) bath (4 mg/ml 1 DAPI (Sigma) in PBS). Samples were mounted in Vectashield (Vector™). Confocal image were acquired as stacks every 0.5 μm for a total of 1.5 μm using a Leica SP5-MP confocal microscope and maximum projections were done with the LAS -AF software. Telomere signal intensity was quantified using Definiens software.

High throughput (HT)-Q-FISH on peripheral blood leukocytes was done as described (Canela et al., 2007a). Briefly, 120-150 μl of blood were extracted from the facial vein. Erythrocytes were lysed (Erythrocyte lysis buffer, Qiagen) and 30-90 k leukocytes were plated in duplicate into clear-bottom, black-walled 96-well plates pre-coated for 30 min with 0.001% poly-L-lysine. Plates were incubated at 37° C. for 2 h and fixed with methanol/acetic acid (3:1, v/v) 2×10 min and overnight at −20° C. Fixative was removed, plates dried for at least 1 h at 37° C. and samples were rehydrated in PBS. Plates were then subjected to a standard Q-FISH protocol (see above) using a telomere-specific PNA-CY3 probe; DAPI was used to stain nuclei. Sixty images per well were captured using the OPERA (Perkin Elmer) High-Content Screening system. TL values were analysed using individual telomere spots (>10,000 telomere spots per sample). The average fluorescence intensities of each sample were converted into kilobase using L5178-R and L5178-S cells as calibration standards, which have stable TLs of 79.7 and 10.2 kb, respectively. Samples were analyzed in duplicate.

### AAV9-Tert Targets Bone Marrow and Hematopoietic Stem Cells

First, we set out to address the capability of AAV9 vectors to transduce the bone marrow upon intra-venous injection by using both a AAV9-EGFP reporter virus, which allows determination of the location and percentage of AAV9-transduced cells, as well as by determining Tert mRNA expression in vivo in different bone marrow cell populations following AAV9-Tert treatment. To this end, we first injected wild-type mice with AAV9-EGFP particles at a concentration of 3.5E12 viral genomes per mouse through tail vein injections. Immunohistochemistry analysis of bone marrow section with specific anti-EGFP antibodies revealed 2% positive EGFP expressing cells in the middle bone sections and this was increased up to 10% in the regions adjacent to the joints, which were the ones showing the highest AAV9-transduction. We then injected wild-type mice with the same amount of AAV9-Tert particles and determined Tert mRNA expression by RT-PCR in whole bone marrow isolates at two weeks and 8 months after virus injection. As soon as two weeks post-treatment with the AAV9 vectors, we found increased Tert mRNA expression in the AAV9-Tert treated mice compared to those treated with the AAV9 empty vector and this difference was maintained still 8 months after initial treatment. We then studied Tert mRNA expression specifically in the blood-forming cells of the bone marrow. To this end, we performed FACS sorting of c -kit and Sca-1 positive HSCs cells and lin-positive lineage committed cells. We found a significant increase in both

HSCs (10 fold) and lineage committed bone marrow cells (3.5 fold) in Tert mRNA in AAV9-Tert treated mice compared to mice treated with the empty vector, demonstrating that bone marrow cells including HSCs cells are targeted by Tert gene therapy. Given that we achieved increased Tert expression in HSCs, we next addressed whether this affected their stem cell potential. To this end, we performed a colony forming cell assay (MethoCult) Interestingly, we observed significantly increased number of colonies in the AAV9-Tert mice compared to the empty vector controls.

In summary these data suggest that AAV9 administered at a high dose can target hematopoietic cells and that these enhances the proliferation capacity of those cells.

### AAV9-Tert Treatment in a Mouse Model of Aplastic Anemia Resecues Survival

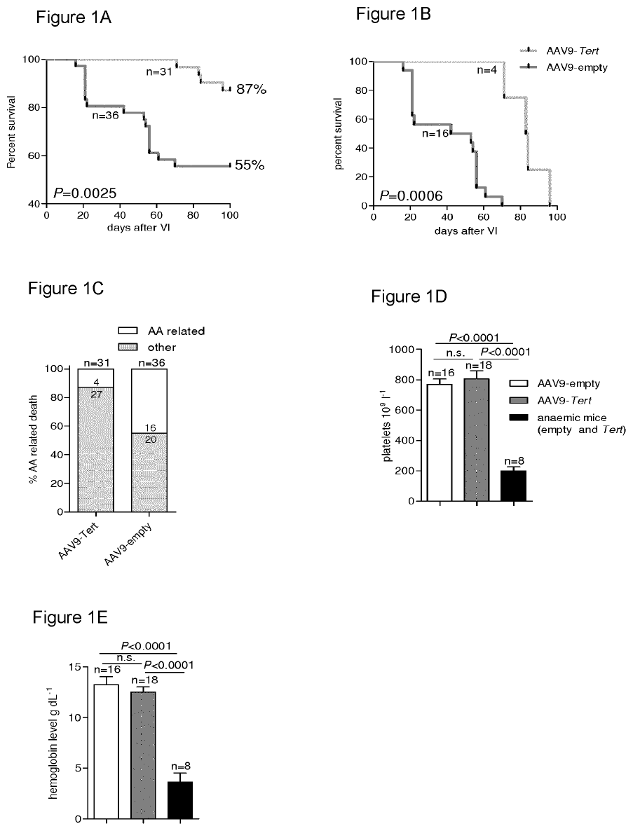

We next tested whether treatment with AAV9-Tert is effective in increasing survival upon induction of lethal aplastic anemia owing to critically short telomeres (Beier et al., 2012). In particular, we used a conditional Trf1 mouse model recently developed by us in which we lethally irradiate wild-type mice and transplant them with bone marrow isolated from Trf1^{lox/lox} Mx1-Cre mice to exclusively study the effects on bone marrow. Trf1 deletion can be induced by administration of pl:pC and subsequent expression of the Cre recombinase (Beier et al., 2012). Cells depleted for Trf1 die and are rapidly removed from the bone marrow, while cells that remain with intact Trf1 undergo compensatory rounds of cell division which leads to rapid telomere shortening, follow by replicative senescence and finally results in bone marrow failure. In the specific experimental settings here we induced Trf1 deletion by injecting mice 3 times per week with pI:pC for a total period of 5 weeks, at which point these mice start showing signs of aplastic anemia (Beier et al., 2012). One week after we stopped the induction of Trf1 deletion, mice were subjected to gene therapy treatment with AAV9-Tert or AAV9-empty control vectors. We monitored the survival of these mice for 100 days following the treatment with the AAV9 vectors. Strikingly, AAV9-Tert treatment drastically improved survival (87%) compared with mice treated with the empty vector (55%) (FIG. 1A). In particular, while only 4 mice injected with AAV9-Tert developed aplastic anemia during this time (13%), 16 mice of the control group (44%) died with clear signs of aplastic anemia (FIG. 1B, C). In agreement with the anemic appearance blood count analysis from these mice (blood drawn from AAV9-empty and AAV9-Tert upon sacrificing) showed a drastic drop in platelet count and haemoglobin level compared with mice without signs of aplastic anemia (FIG. 1D, E). Post mortem histopathologic analysis of bone marrow sections from mice that died during the first 100 days further confirmed the aplastic anemia phenotype. In particular, mice presented with severe bone marrow hypo- and aplasia in 2 or all 3 blood lineages. While the diagnosis at the point of death in both groups was marrow bone failure and aplasia the phenotype appeared milder in the AAV9-Tert group compared with the AAV9-empty group as seen by higher bone marrow cellularity.

Our results suggest that AAV9-Tert gene therapy significantly reduces aplastic anemia mortality by preventing the loss of blood forming hematopoietic cells.

### Telomerase Treatment Leads to Telomere Elongation in Peripheral Blood and Bone Marrow

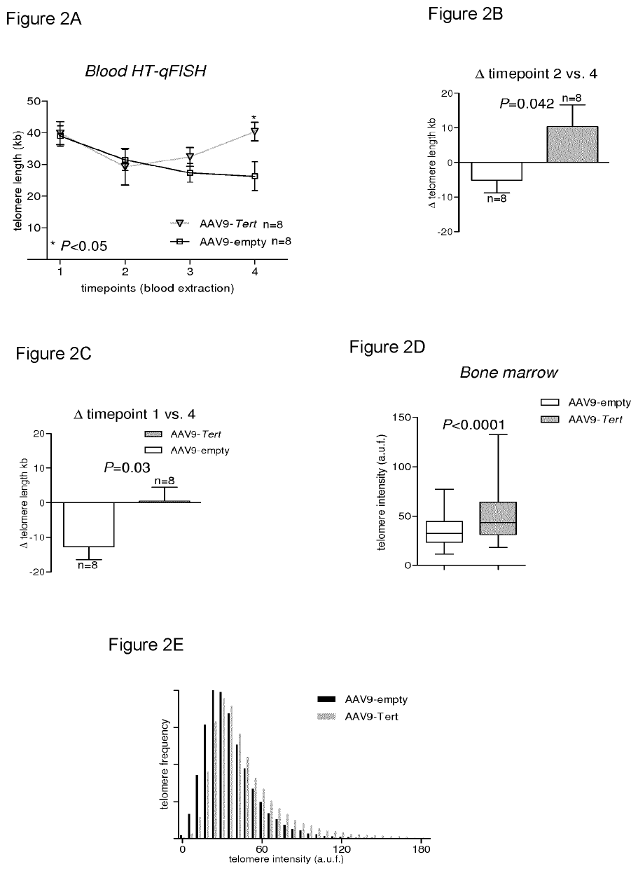

Because the aplastic anemia phenotype in our mouse model is caused by the loss of telomeres, we next compared telomere length in mice treated with telomerase to mice receiving the control vector. First we used HT-Q-FISH technology (Canela et al., 2007b) to follow telomere length in peripheral blood monocytes in a longitudinal manner To do so, we extracted blood at 4 different time points; after bone marrow engraftment (1), after pLpC treatment (2), 2 months after AAV9 injection (3) and 4 months after AAV9 injection (4). As expected we found that telomere length between time point 1 and 2 in both groups drops by approximately 10 kb which is owed to the pI:pC treatment. While telomere length in the AAV9-empty group between time point 2 and 4 continuous to slightly shorten, AAV9-Tert treatment led to a net increase in average telomere of 10 kb (FIG. 2A, B). Throughout the course of this experiment AAV9-empty treated mice showed an average telomere length loss of 12 kb, whereas in AAV9-Tert treated mice telomeres were re-elongated to similar levels as before the pI:pC treatment (FIG. 2C). Next we performed Q-FISH analysis on bone marrow cross-sections. In agreement with longer telomere length in peripheral blood we found that AAV9-Tert treated mice had significantly longer telomeres compared with empty vector treated mice (FIG. 2D, E).

### REFERENCES

ADDIN EN.REFLIST Armanios, M. (2012). An emerging role for the conserved telomere component 1 (CTC1) in human genetic disease. Pediatr Blood Cancer 59, 209-210.

Armanios, M., and Blackburn, E. H. (2012). The telomere syndromes. Nature reviews. Genetics 13, 693-704.

Ayuso, E., Mingozzi, F., Montane, J., Leon, X., Anguela, X. M., Haurigot, V., Edmonson, S. A., Africa, L., Zhou, S., High, K. A., et al. (2010). High AAV vector purity results in serotype- and tissue-independent enhancement of transduction efficiency. Gene therapy 17, 503-510.

Beier, F., Foronda, M., Martinez, P., and Blasco, M. A. (2012). Conditional TRF1 knockout in the hematopoietic compartment leads to bone marrow failure and recapitulates clinical features of Dyskeratosis congenita. Blood.

Bernardes de Jesus, B., Vera, E., Schneeberger, K., Tejera, A. M., Ayuso, E., Bosch, F., and Blasco, M. A. (2012). Telomerase gene therapy in adult and old mice delays aging and increases longevity without increasing cancer. EMBO molecular medicine 4, 691-704.

Bernardes de Jesus de Jesus, B. and Blasco, M. A, (2013). Telomerase at the intersection of cancer and aging. Trends Genet. 29, 513-520.

Blasco, M. A. (2007). Telomere length, stem cells and aging. Nature chemical biology 3, 640-649.

Blasco, M. A., Lee, H. W., Hande, M. P., Samper, E., Lansdorp, P. M., DePinho, R. A., and Greider, C. W. (1997). Telomere shortening and tumor formation by mouse cells lacking telomerase RNA. Cell 91, 25-34.

Buning, H., Perabo, L., Coutelle, O., Quadt-Humme, S., and Hallek, M. (2008). Recent developments in adeno-associated virus vector technology. The journal of gene medicine 10,717 -733.

Calado, R. T., Yewdell, W. T., Wilkerson, K. L., Regal, J. A., Kajigaya, S., Stratakis, C. A., and Young, N. S. (2009). Sex hormones, acting on the TERT gene, increase telomerase activity in human primary hematopoietic cells. Blood 114, 2236-2243.

Callen, E., Samper, E., Ramirez, M. J., Creus, A., Marcos, R., Ortega, J. J., Olive, T., Badell, I., Blasco, M. A., and Surralles, J. (2002). Breaks at telomeres and TRF2-independent end fusions in Fanconi anemia. Hum Mol Genet 11, 439-444.

Carroll, K. A., and Ly, H. (2009). Telomere dysfunction in human diseases: the long and short of it! International journal of clinical and experimental pathology 2, 528-543.

Dokal, I. (2011). Dyskeratosis congenita. Hematology/the Education Program of the American Society of Hematology. American Society of Hematology. Education Program 2011, 480-486.

Dokal, I., and Vulliamy, T. (2010). Inherited bone marrow failure syndromes. Haematologica 95, 1236-1240.

Duque, S., Joussemet, B., Riviere, C., Marais, T., Dubreil, L., Douar, A. M., Fyfe, J., Moullier, P., Colle, M. A., and Barkats, M. (2009). Intravenous administration of self-complementary AAV9 enables transgene delivery to adult motor neurons. Molecular therapy: the journal of the American Society of Gene Therapy 17, 1187-1196.

Foust, K. D., Nurre, E., Montgomery, C. L., Hernandez, A., Chan, C. M., and Kaspar, B. K. (2009). Intravascular AAV9 preferentially targets neonatal neurons and adult astrocytes. Nat Biotechnol 27, 59-65.

Gadalla, S. M., Cawthon, R., Giri, N., Alter, B. P., and Savage, S. A. (2010). Telomere length in blood, buccal cells, and fibroblasts from patients with inherited bone marrow failure syndromes. Aging (Albany NY) 2, 867-874.

Gao, G. P., Alvira, M. R., Wang, L., Calcedo, R., Johnston, J., and Wilson, J. M. (2002). Novel adeno-associated viruses from rhesus monkeys as vectors for human gene therapy. Proc Natl Acad Sci U S A 99, 11854-11859.

Gonzalez-Suarez, E., Samper, E., Ramirez, A., Flores, J. M., Martin-Caballero, J., Jorcano, J. L., and Blasco, M. A. (2001). Increased epidermal tumors and increased skin wound healing in transgenic mice overexpressing the catalytic subunit of telomerase, mTERT, in basal keratinocytes. EMBO J 20, 2619-2630.

Herrera, E., Samper, E., Martin-Caballero, J., Flores, J. M., Lee, H. W., and Blasco, M. A. (1999). Disease states associated with telomerase deficiency appear earlier in mice with short telomeres. EMBO J 18, 2950-2960.

Holme, H., Hossain, U., Kirwan, M., Walne, A., Vulliamy, T., and Dokal, I. (2012). Marked genetic heterogeneity in familial myelodysplasia/acute myeloid leukaemia. British journal of haematology 158, 242-248.

Inagaki, K., Fuess, S., Storm, T. A., Gibson, G. A., McTiernan, C. F., Kay, M. A., and Nakai, H. (2006). Robust systemic transduction with AAV9 vectors in mice: efficient global cardiac gene transfer superior to that of AAV8. Molecular therapy: the journal of the American Society of Gene Therapy 14, 45-53.

Jaime-Perez, J. C., Colunga-Pedraza, P. R., Gomez-Ramirez, C. D., Gutierrez-Aguirre, C. H., Cantu-Rodriguez, O. G., Tarin-Arzaga, L. C., and Gomez-Almaguer, D. (2011). Danazol as first-line therapy for aplastic anemia. Annals of hematology 90, 523-527.

Jaskelioff, M., Muller, F. L., Paik, J. H., Thomas, E., Jiang, S., Adams, A. C., Sahin, E., Kost-Alimova, M., Protopopov, A., Cadinanos, J., et al. (2011). Telomerase reactivation reverses tissue degeneration in aged telomerase-deficient mice. Nature 469, 102-106.

Jiang, H., Lillicrap, D., Patarroyo-White, S., Liu, T., Qian, X., Scallan, C. D., Powell, S., Keller, T., McMurray, M., Labelle, A., et al. (2006). Multiyear therapeutic benefit of AAV serotypes 2, 6, and 8 delivering factor VIII to hemophilia A mice and dogs. Blood 108, 107-115.

Kaplitt, M. G. (2009). Gene therapy clinical trials in the human brain. Protocol development and review of current applications. Frontiers of neurology and neuroscience 25, 180-188.

Kee, Y., and D'Andrea, A. D. (2012). Molecular pathogenesis and clinical management of Fanconi anemia. J Clin Invest 122, 3799-3806.

Lee, H. W., Blasco, M. A., Gottlieb, G. J., Homer, J. W., 2nd, Greider, C. W., and DePinho, R. A. (1998). Essential role of mouse telomerase in highly proliferative organs. Nature 392, 569-574.

Maguire, A. M., Simonelli, F., Pierce, E. A., Pugh, E. N., Jr., Mingozzi, F., Bennicelli, J., Banfi, S., Marshall, K. A., Testa, F., Surace, E. M., et al. (2008). Safety and efficacy of gene transfer for Leber's congenital amaurosis. The New England journal of medicine 358, 2240-2248.

Manno, C. S., Pierce, G. F., Arruda, V. R., Glader, B., Ragni, M., Rasko, J. J., Ozelo, M. C., Hoots, K., Blatt, P., Konkle, B., et al. (2006). Successful transduction of liver in hemophilia by AAV-Factor IX and limitations imposed by the host immune response. Nature medicine 12, 342-347.

Martinez, P., and Blasco, M. A. (2011). Telomeric and extra-telomeric roles for telomerase and the telomere-binding proteins. Nature reviews. Cancer 11, 161-176.

Mas, A., Montane, J., Anguela, X. M., Munoz, S., Douar, A. M., Riu, E., Otaegui, P., and Bosch, F. (2006). Reversal of type 1 diabetes by engineering a glucose sensor in skeletal muscle. Diabetes 55, 1546-1553.

Mason, P. J., and Bessler, M. (2011). The genetics of dyskeratosis congenita. Cancer genetics 204, 635-645.

Matsushita, T., Elliger, S., Elliger, C., Podsakoff, G., Villarreal, L., Kurtzman, G. J., Iwaki, Y., and Colosi, P. (1998). Adeno-associated virus vectors can be efficiently produced without helper virus. Gene therapy 5, 938-945.

Mavilio, F. (2012). Gene therapies need new development models. Nature 490,7.

Niemeyer, G. P., Herzog, R. W., Mount, J., Arruda, V. R., Tillson, D. M., Hathcock, J., van Ginkel, F. W., High, K. A., and Lothrop, C. D., Jr. (2009). Long-term correction of inhibitor-prone hemophilia B dogs treated with liver-directed AAV2-mediated factor IX gene therapy. Blood 113, 797-806.

O'Reilly, M., Shipp, A., Rosenthal, E., Jambou, R., Shih, T., Montgomery, M., Gargiulo, L., Patterson, A., and Corrigan-Curay, J. (2012). NIH oversight of human gene transfer research involving retroviral, lentiviral, and adeno-associated virus vectors and the role of the NIH recombinant DNA advisory committee. Methods in enzymology 507, 313-335.

Samper, E., Flores, J. M., and Blasco, M. A. (2001). Restoration of telomerase activity rescues chromosomal instability and premature aging in Terc−/− mice with short telomeres. EMBO Rep 2, 800-807.

Savage, S. A., and Alter, B. P. (2008). The role of telomere biology in bone marrow failure and other disorders. Mechanisms of ageing and development 129, 35-47.

Savage, S. A., Calado, R. T., Xin, Z. T., Ly, H., Young, N. S., and Chanock, S. J. (2006). Genetic variation in telomeric repeat binding factors 1 and 2 in aplastic anemia. Experimental hematology 34, 664-671.

Stroes, E. S., Nierman, M. C., Meulenberg, J. J., Franssen, R., Twisk, J., Henny, C. P., Maas, M. M., Zwinderman, A. H., Ross, C., Aronica, E., et al. (2008). Intramuscular administration of AAV1-lipoprotein lipase S447X lowers triglycerides in lipoprotein lipase-deficient patients. Arteriosclerosis, thrombosis, and vascular biology 28, 2303-2304.

Tafuro, S., Ayuso, E., Zacchigna, S., Zentilin, L., Moimas, S., Dore, F., and Giacca, M. (2009). Inducible adeno-associated virus vectors promote functional angiogenesis in adult organisms via regulated vascular endothelial growth factor expression. Cardiovascular research 83, 663-671.

Tomas-Loba, A., Flores, I., Fernandez-Marcos, P. J., Cayuela, M. L., Maraver, A., Tejera, A., Borras, C., Matheu, A., Klatt, P., Flores, J. M., et al. (2008). Telomerase reverse transcriptase delays aging in cancer-resistant mice. Cell 135, 609-622.

Walne, A. J., Vulliamy, T., Beswick, R., Kirwan, M., and Dokal, I. (2008). TINF2 mutations result in very short telomeres: analysis of a large cohort of patients with dyskeratosis congenita and related bone marrow failure syndromes. Blood 112, 3594-3600.

Yamaguchi, H., Baerlocher, G. M., Lansdorp, P. M., Chanock, S. J., Nunez, O., Sloand, E., and Young, N. S. (2003). Mutations of the human telomerase RNA gene (TERC) in aplastic anemia and myelodysplastic syndrome. Blood 102, 916-918.

Yamaguchi, H., Calado, R. T., Ly, H., Kajigaya, S., Baerlocher, G. M., Chanock, S. J., Lansdorp, P. M., and Young, N. S. (2005). Mutations in TERT, the gene for telomerase reverse transcriptase, in aplastic anemia. The New England journal of medicine 352, 1413-1424.

Ziegler, P., Schrezenmeier, H., Akkad, J., Brassat, U., Vankann, L , Panse, J., Wilop, S., Balabanov, S., Schwarz, K., Martens, U. M., and Brummendorf, T. H. (2012). Telomere elongation and clinical response to androgen treatment in a patient with aplastic anemia and a heterozygous hTERT gene mutation. Annals of hematology 91, 1115-1120.

Ayuso, E., V. Blouin, M. Lock, S. McGorray, X. Leon, M. R. Alvira, A. Auricchio, S. Bucher, A. Chtarto, K. R. Clark, C. Darmon, M. Doria, W. Fountain, G. Gao, K. Gao, M. Giacca, J. Kleinschmidt, B. Leuchs, C. Melas, H. Mizukami, M. Muller, Y. Noordman, O. Bockstael, K. Ozawa, C. Pythoud, M. Sumaroka, R. Surosky, L. Tenenbaum, I. Van der Linden, B. Weins, J. F. Wright, X. Zhang, L. Zentilin, F. Bosch, R. O. Snyder & P. Moullier, (2014) Manufacturing and Characterization of a Recombinant Adeno-Associated Virus Type 8 Reference Standard Material. Hum Gene Ther.

Ayuso, E., F. Mingozzi, J. Montane, X. Leon, X. M. Anguela, V. Haurigot, S. A. Edmonson, L. Africa, S. Zhou, K. A. High, F. Bosch & J. F. Wright, (2010) High AAV vector purity results in serotype- and tissue-independent enhancement of transduction efficiency. Gene Ther 17: 503-510.

Ball, S. E., F. M. Gibson, S. Rizzo, J. A. Tooze, J. C. Marsh & E. C. Gordon-Smith, (1998) Progressive telomere shortening in aplastic anemia. Blood 91: 3582-3592.

Beier, F., M. Foronda, P. Martinez & M. A. Blasco, (2012) Conditional TRF1 knockout in the hematopoietic compartment leads to bone marrow failure and recapitulates clinical features of Dyskeratosis congenita. Blood.

Bernardes de Jesus, B., E. Vera, K. Schneeberger, A. M. Tejera, E. Ayuso, F. Bosch & M. A. Blasco, (2012) Telomerase gene therapy in adult and old mice delays aging and increases longevity without increasing cancer. EMBO Mol Med 4: 1-14.

Blackburn, E. H., (2001) Switching and signaling at the telomere. Cell 106: 661-673.

Canela, A., P. Klatt & M. A. Blasco, (2007a) Telomere length analysis. Methods Mol Biol 371: 45-72.

Canela, A., E. Vera, P. Klatt & M. A. Blasco, (2007b) High-throughput telomere length quantification by FISH and its application to human population studies. Proc Natl Acad Sci U S A 104: 5300-5305.

de Lange, T., (2005) Shelterin: the protein complex that shapes and safeguards human telomeres. Genes Dev 19: 2100-2110.

Dokal, I., (2011) Dyskeratosis congenita. Hematology Am Soc Hematol Educ Program 2011: 480-486.

Dokal, I. & T. Vulliamy, (2010) Inherited bone marrow failure syndromes. Haematologica 95: 1236-1240.

Flores, I., A. Canela, E. Vera, A. Tejera, G. Cotsarelis & M. A. Blasco, (2008) The longest telomeres: a general signature of adult stem cell compartments. Genes Dev 22: 654-667.

Flores, I., M. L. Cayuela & M. A. Blasco, (2005) Effects of telomerase and telomere length on epidermal stem cell behavior. Science 309: 1253-1256.

Harley, C. B., A. B. Futcher & C. W. Greider, (1990) Telomeres shorten during ageing of human fibroblasts. Nature 345: 458-460.

Hiyama, E. & K. Hiyama, (2007) Telomere and telomerase in stem cells. Br J Cancer 96: 1020-1024.

Maciejewski, J. P., S. Anderson, P. Katevas & N. S. Young, (1994) Phenotypic and functional analysis of bone marrow progenitor cell compartment in bone marrow failure. Br J Haematol 87: 227-234.

Marsh, J. C., S. E. Ball, J. Cavenagh, P. Darbyshire, I. Dokal, E. C. Gordon-Smith, J. Keidan, A. Laurie, A. Martin, J. Mercieca, S. B. Killick, R. Stewart & J. A. Yin, (2009) Guidelines for the diagnosis and management of aplastic anaemia. Br J Haematol 147: 43-70.

Martinez, P., M. Thanasoula, P. Munoz, C. Liao, A. Tejera, C. McNees, J. M. Flores, O. Fernandez-Capetillo, M. Tarsounas & M. A. Blasco, (2009) Increased telomere fragility and fusions resulting from TRF1 deficiency lead to degenerative pathologies and increased cancer in mice. Genes Dev 23: 2060-2075.

Matsushita, T., S. Elliger, C. Elliger, G. Podsakoff, L. Villarreal, G. J. Kurtzman, Y. Iwaki & P. Colosi, (1998) Adeno-associated virus vectors can be efficiently produced without helper virus. Gene Ther 5: 938-945.

Nakao, S., (1997) Immune mechanism of aplastic anemia. Int J Hematol 66: 127-134.

Samper, E., P. Fernandez, R. Eguia, L. Martin-Rivera, A. Bernad, M. A. Blasco & M. Aracil, (2002) Long-term repopulating ability of telomerase-deficient murine hematopoietic stem cells. Blood 99: 2767-2775.

Samper, E., F. A. Goytisolo, P. Slijepcevic, P. P. van Buul & M. A. Blasco, (2000) Mammalian Ku86 protein prevents telomeric fusions independently of the length of TTAGGG repeats and the G-strand overhang. EMBO Rep 1: 244-252.

Scopes, J., M. Bagnara, E. C. Gordon-Smith, S. E. Ball & F. M. Gibson, (1994) Haemopoietic progenitor cells are reduced in aplastic anaemia. Br J Haematol 86: 427-430.

Vulliamy, T., A. Marrone, I. Dokal & P. J. Mason, (2002) Association between aplastic anaemia and mutations in telomerase RNA. Lancet 359: 2168-2170.

Wynn, R. F., M. A. Cross, C. Hatton, A. M. Will, L. S. Lashford, T. M. Dexter & N. G. Testa, (1998) Accelerated telomere shortening in young recipients of allogeneic bone-marrow transplants. Lancet 351: 178-181.

1. A method of treating a disease or condition associated with shortened telomere length, the method comprising: administering to a subject in need thereof a recombinant viral vector comprising at least a capsid and a nucleic acid encoding telomerase reverse transcriptase (TERT) operably linked to a regulatory sequence driving the expression of the encoded TERT.

2. The method of claim 1, wherein TERT is encoded by a nucleic acid sequence comprising the sequence of SEQ ID NO: 1 or SEQ ID NO: 3.

3. The method of claim 1, wherein TERT comprises an amino acid sequence of SEQ ID NO:2 or SEQ ID NO: 4.

4. (canceled)

5. The method of claim 1, wherein the recombinant viral vector is a non-integrative vector.

6. The method of claim 1, wherein the recombinant viral vector is an adeno-associated virus-based non-integrative vector.

7. The method of claim 1, wherein the recombinant viral vector is an adeno-associated virus-based vector derived from a serotype 9 adeno-associated virus (AAV9).

8. The method of claim 7, wherein the capsid of the adeno-associated virus-based vector is made of capsid proteins of the serotype 9 adeno-associated virus (AAV9), and the nucleic acid sequence contained in the capsid is flanked at both ends by internal terminal repeats corresponding to serotype 2 adeno-associated viruses.

9. The method of claim 8, wherein the nucleic acid contained in the capsid comprises a fragment which encodes the amino acid sequence coding for TERT.

10. The method of claim 1, wherein the vector comprises a regulatory sequence which is a constitutive promoter.

11. The method of claim 10, wherein the regulatory sequence is the cytomegalovirus (CMV) promoter.

12. The method of claim 1, wherein the condition associated with short telomere length is characterized by mutations in a gene or genes involved in telomere maintenance.

13. The method of claim 1, wherein the disease or condition associated with shortened telomere length is selected from the group consisting of pulmonary fibrosis, dyskeratosis congenita, aplastic anaemia, myelodysplastic syndrome, and Fanconi anaemia.


SEQUENCE LISTING (26 sequences)

SEQ ID NO: 1 - DNA, 4018 bp, Homo sapiens
  source 1..4018: mol_type = other DNA
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 3982 bp, Homo sapiens
  source 1..3982: mol_type = other DNA
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 4 - protein, 1120 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 5 - DNA, 3426 bp, Mus musculus
  source 1..3426: mol_type = other DNA
gtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatg

SEQ ID NO: 6 - protein, 1122 aa, Mus musculus
MTRAPRCPAVRSLLRSRYREVWPLATFVRRLGPEGRRLVQPGDPKIYRTLVAQCLVCMHWGSQPPPADLSFHQVSSLKELVARVVQRLCERNERNVLAFGFELLNEARGGPPMAFTSSVRSYLPNTVIETLRVSGAWMLLLSRVGDDLLVYLLAHCALYLLVPPSCAYQVCGSPLYQICATTDIWPSVSASYRPTRPVGRNFTNLRFLQQIKSSSRQEAPKPLALPSRGTKRHLSLTSTSVPSAKKARCYPVPRVEEGPHRQVLPTPSGKSWVPSPARSPEVPTAEKDLSSKGKVSDLSLSGSVCCKHKPSSTSLLSPPRQNAFQLRPFIETRHFLYSRGDGQERLNPSFLLSNLQPNLTGARRLVEIIFLGSRPRTSGPLCRTHRLSRRYWQMRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 7 - DNA, 21 bp, synthetic construct
  misc_feature 1..21: note = Primer
  source 1..21: mol_type = other DNA
ggcaccacaccttctacaatg

SEQ ID NO: 8 - DNA, 18 bp, synthetic construct
  misc_feature 1..18: note = Primer
  source 1..18: mol_type = other DNA
gtggtggtgaagctgtag

SEQ ID NO: 9 - DNA, 18 bp, synthetic construct
  misc_feature 1..18: note = Primer
  source 1..18: mol_type = other DNA
ggattgccactggctccg

SEQ ID NO: 10 - DNA, 21 bp, synthetic construct
  misc_feature 1..21: note = Primer
  source 1..21: mol_type = other DNA
tgcctgacctcctcttgtgac

SEQ ID NO: 11 - DNA, 20 bp, synthetic construct
  misc_feature 1..20: note = Primer
  source 1..20: mol_type = other DNA
cgtaccccgattcaggtgat

SEQ ID NO: 12 - DNA, 22 bp, synthetic construct
  misc_feature 1..22: note = Primer
  source 1..22: mol_type = other DNA
ttgagcagaagagctgctacgt

SEQ ID NO: 13 - DNA, 21 bp, synthetic construct
  misc_feature 1..21: note = Primer
  source 1..21: mol_type = other DNA
ggcaaagtggagaggatcgac

SEQ ID NO: 14 - DNA, 18 bp, synthetic construct
  misc_feature 1..18: note = Primer
  source 1..18: mol_type = other DNA
tcgtggctgttgcgtagg

SEQ ID NO: 15 - DNA, 19 bp, synthetic construct
  misc_feature 1..19: note = Primer
  source 1..19: mol_type = other DNA
tgcgccctccgtatcttac

SEQ ID NO: 16 - DNA, 22 bp, synthetic construct
  misc_feature 1..22: note = Primer
  source 1..22: mol_type = other DNA
atcttagaggccacgaacatgc

SEQ ID NO: 17 - DNA, 23 bp, synthetic construct
  misc_feature 1..23: note = Primer
  source 1..23: mol_type = other DNA
cagcctactggagatcaggatga

SEQ ID NO: 18 - DNA, 22 bp, synthetic construct
  misc_feature 1..22: note = Primer
  source 1..22: mol_type = other DNA
ggagtccttggatgagtctcga

SEQ ID NO: 19 - DNA, 24 bp, synthetic construct
  misc_feature 1..24: note = Primer
  source 1..24: mol_type = other DNA
gcgaactcacacaggcgagaaacc

SEQ ID NO: 20 - DNA, 23 bp, synthetic construct
  misc_feature 1..23: note = Primer
  source 1..23: mol_type = other DNA
tcgcttcctcttcctccgacaca

SEQ ID NO: 21 - DNA, 19 bp, synthetic construct
  misc_feature 1..19: note = Primer
  source 1..19: mol_type = other DNA
cccattgcccctgctcctg

SEQ ID NO: 22 - DNA, 19 bp, synthetic construct
  misc_feature 1..19: note = Primer
  source 1..19: mol_type = other DNA
tgtgtccgccggtgtctgg

SEQ ID NO: 23 - DNA, 26 bp, synthetic construct
  misc_feature 1..26: note = Primer
  source 1..26: mol_type = other DNA
caattacggggtcattagttcatagc

SEQ ID NO: 24 - DNA, 26 bp, synthetic construct
  misc_feature 1..26: note = Primer
  source 1..26: mol_type = other DNA
atacgtagatgtactgccaagtagga

SEQ ID NO: 25 - DNA, 21 bp, synthetic construct
  misc_feature 1..21: note = Primer
  source 1..21: mol_type = other DNA
cttcctgccacaatgtcacag

SEQ ID NO: 26 - DNA, 23 bp, synthetic construct
  misc_feature 1..23: note = Primer
  source 1..23: mol_type = other DNA
cctttctcatgcttgcttctctg